(19) United States
(12) Patent Application Publication
(10) Pub. No.: US 2010/0050316 A1
(43) Pub. Date: Mar. 4, 2010

(54) SYNTHETIC WOVEN PATIENT GOWN FOR PREVENTING AND REDUCING SKIN WOUNDS

(75) Inventors: W. ALLEN LEONARD, Greensboro, NC (US); Neil Blanton, Oak Ridge, NC (US); Terry Montgomery, Greensboro, NC (US); Jones McCall, Greensboro, NC (US)

Correspondence Address: MACCORD MASON PLLC, 300 N. GREENE STREET, SUITE 1600, P. O. BOX 2974, GREENSBORO, NC, 27402, US

(73) Assignee: Precision Fabrics Group, Inc.

(21) Appl. No.: 12/501,915

(22) Filed: Jul. 13, 2009

Related U.S. Application Data
(63) Continuation-in-part of application No. 11/152,914, filed on Jun. 13, 2005.
Application No. 11/152,914 is a continuation-in-part of application No. 10/985,739, filed on Nov. 10, 2004, now abandoned.

(51) Int. Cl. A41D 13/12; A61F 13/00; D05B 23/00; A61M 35/00

(52) U.S. Cl. 2/114; 128/888; 112/475.09; 604/289

(57) ABSTRACT

A patient gown comprised of therapeutic fabric for the prevention and treatment of skin wounds, and pressure wounds in particular, which includes a woven fabric having warp yarns and filling yarns woven to provide a smooth fabric surface. One of the warp or filling yarns is at least about 40% by weight of the fabric of continuous filament nylon, and the other of the warp or filling yarns is from about 0% to about 60% by weight of the fabric of continuous filament polyester or nylon having non-round filament cross sections. The fabric includes a conductive yarn at about 1% to about 2% by weight to control static dissipation, and an antimicrobial substance is topically applied or inherently available in the fabric. A method of using the patient gown to prevent and treat skin wounds, and pressure wounds in particular, is also disclosed.

### BACKGROUND OF THE INVENTION

The present invention relates generally to therapeutic fabrics, and more specifically to articles that include a woven fabric having advantageous antimicrobial, anti-static, wicking and drying properties, to aid in the prevention and treatment of dermatitis, skin wounds, pressure wounds and other skin ailments. Similar fabrics for therapeutic skin care bedding are disclosed in co-pending U.S. patent application Ser. No. 10/985,739 filed Nov. 10, 2004, and in co-pending U.S. patent application Ser. No. 11/152,914 filed Jun. 13, 2005, the entire disclosures of which are hereby incorporated by reference. Medical research in support of the technology disclosed in these applications has been published in Pediatric Dermatology, Vol. 25, No. 4, 439-433, July/August 2008, in an article entitled “Use of a Silklike Bedding Fabric in Patients with Atopic Dermatitis”, the entire disclosure of which is hereby incorporated by reference. On Jan. 25, 2007, the U.S. Food and Drug Administration granted 510(k) clearance as a Class 1 medical device (registration number K061242), the entire disclosure of which is hereby incorporated by reference. Moreover, two applications filed contemporaneously with this application, U.S. patent application Ser. No. ______ filed ______, and U.S. patent application Ser. No. ______ filed ______, pertaining to patient gowns and bedding underpads respectively, are hereby incorporated by reference.

A significant portion of the world's population is afflicted with skin problems. Nearly 15 million Americans have eczema, a chronic skin condition marked by itchy, red patches of inflamed skin. Six to seven million Americans have psoriasis, which is a skin disease that causes unsightly sores and skin scales. Such skin diseases account for a large portion of annual healthcare costs. For instance, psoriasis accounts for about $3 billion dollars a year in healthcare costs. Of course this doesn't include non-financial costs such as time spent in treatment, sleep deprivation, intractable itching, skin damage, soreness, the social stigma of a visible skin disease, and inconvenience associated with treatment, avoidance of certain clothing, using special detergents on clothing and bedding, and controlling environmental conditions to maintain the proper level of moisture on the affected skin.

Likewise, skin wounds, and in particular pressure wounds, continue to be a growing problem for healthcare systems around the world, particularly concerning the population which is aged, bedridden, unconscious, unable to sense pain, or immobile. Pressure wounds, also known as bedsores, are areas of localized tissue destruction caused by the compression of soft tissue between a bony prominence and an external surface for a prolonged period of time. They develop when blood supply to the skin is cut off for more than two to three hours. As the skin dies, the pressure wound starts as a red, painful area, which eventually turns purple. They often occur on areas of the skin that are under pressure from the patient lying in bed, sitting in a wheelchair, and/or wearing a cast for a prolonged period of time. Common areas include the buttocks (on the sacrum or iliac crest), or the heels of the feet. An untreated wound can break open and become infected, and/or extend deep into the muscle. Once a pressure wound develops, it is often very slow healing. Known methods of prevention include inspecting the skin for areas of redness (the first sign of skin breakdown), frequent turning and repositioning, providing soft padding in wheelchairs and beds to reduce pressure, and/or providing good skin care by keeping the skin clean and dry. Treatment may include removing pressure on the affected area, protecting the wound with medicated gauze or other special dressings, keeping the wound clean, transplanting healthy skin to the wound area and/or medication (i.e., antibiotics to treat infections).

More than 1 million persons in the US develop pressure wounds each year, approximately 7% of which are acquired in a hospital. The average cost of treating a person admitted to the hospital for pressure wounds is $37,288, which translates to approximately $2.2 to $3.6 billion dollars per year spent on treating pressure wounds in an acute care setting. Again, this does not account for the physical or emotion toll on the afflicted, their families and caretakers.

Moisture, friction and shearing can cause or exacerbate skin problems such as dermatitis, eczema, psoriasis and pressure wounds. For example, a person who has been exposed to too much moisture will have vulnerable skin. When this person moves across a coarse moist surface such as a bed sheet, their skin is subjected to shear force injuries caused by friction. Likewise, they can be injured by their apparel, or another item of bedding which is in contact with their skin, particularly where that article has increased frictional force or decreased shear force resistance. The problem is further compounded insofar as skin that has been damaged by moisture, friction and/or shearing is much more likely to develop pressure-related damage such as ulcers or pressure wounds. Moreover, the likelihood of injury increases when the skin is in the presence of biological substances such as urine, stool, perspiration or wound drainage, which may contain factors other than moisture that irritate the skin.

Accordingly, it is not only important to provide bedding and apparel with properties that prevent skin disorders, it is important that the bedding and apparel actually treat disorders that may present themselves. Unfortunately however, fabrics for constructing bedding and apparel to prevent and treat skin disorders, and pressure wounds in particular, are not prevalent in the art.

Hospital linens such as bottom sheets, top sheets, and pillow cases are generally considered by hospitals to be housekeeping commodities, as opposed to therapeutic items. Accordingly, these bedding items are typically constructed of low-tech and low cost fabric, such as conventional cotton-polyester blends. A typical conventional hospital bedding fabric is a plain weave construction of 120 warp yarns per inch and 74 filling yarns per inch, producing a fabric that is 4.12 ounces per square yard. The warp yarns are 39/1 spun yarns, equivalent to 136 denier, in a blend of 50% cotton and 50% staple polyester. The filling yarns are 35.6/1 spun yarns, equivalent to 149 denier, in a blend of 50% cotton and 50% staple polyester. These fabrics have no special finishes, moisture handling characteristics, antimicrobial properties, anti-static characteristics, or soil-release properties. In short, these are low-end bedding products similar to what one might find in a home or hotel, and are not made to prevent or treat skin disorders.

Another bedding item often used in a hospital is an underpad, also known as a protective pad, which provides a means for shielding mattresses from biological fluids that emanate from a person in bed. They range from a very simple sheet of waterproof material such as rubber, to more a complex multi-layered device. They may be intended for one use (disposable), or may be reusable. Underpads are intended to be positioned between the bottom sheet and top sheet of a bed, so a person in the bed is laying directly on the underpad, with gravity carrying the emanating biologicals to the underpad. To varying degrees this protects the bottom sheet and mattress thereunder. Since the underpad is typically in direct contact with the patient, efforts have been made to make the underpad more comfortable. For example, rubberized pads may include flocking to contact the patient, or vinyl backing may include a quilted cloth.

Like sheets, underpads are considered by hospital administration to be housekeeping articles—commodities which are intended to maintain patients' cleanliness and protect furniture, at the lowest possible cost. However, in situations where patients are at risk for the development of pressure wounds, commodity products are insufficient and ineffective. Research indicates that standard hospital-issue commodities actually exacerbate wound development. Specifically, with conventionally gowned patients using conventional bedding, including underpads, the average number of wounds per patient increased 41% between admittance and discharge.

Underpads may comprise an absorbent material that is covered in the conventional hospital bedding fabric discussed above. While an underpad of this construction may effectively protect the mattress, and possibly even be relatively comfortable, it does not rise to the level of being therapeutic insofar as it doesn't prevent or treat skin problems, and pressure wounds in particular.

Another example of a hospital article that is commoditized to the detriment of patients is the patient gown, also known as a hospital gown. These garments are typically constructed of cotton/poly blends, for example the aforementioned conventional bedding fabric. They are typically relatively inexpensive and easy to wash, but do not possess properties which would actually prevent or treat skin disorders, and particularly pressure wounds.

Although the majority of technological advances in the patient gown art are directed towards improved patient coverage for privacy purposes, some progress has been made in treating disease with garments. For example, U.S. Pat. No. 6,868,854 to Kempe teaches a method of treating fibromyalgia with a radiation-shielding gown, and U.S. Pat. No. 7,003,804 to Lewis teaches a surgical gown with a skin wellness agent applied that rubs off onto the wearer.

A line of products marketed under the trade name DermaSilk™ has applications in skin care apparel. Specifically, the DermaSilk technology has shown excellent clinical results in helping to heal atopic dermatitis and psoriasis, as well as skin ulcers that form as a result of diabetes. DermaSilk therapeutic apparel include undergarments and body wraps knitted of 100% silk yarns and treated with a durable antimicrobial agent.

Silk is effective in this case since silk fibers have a chemical structure very similar to that of human hair (97% protein, 3% fat, and wax matter). Silk fibers are perfectly smooth and cylindrical. As such, they do not create mechanical friction with the skin. Further, silk is naturally hygroscopic, absorbing up to 30% of its own weight in sweat without becoming wet. This is important in aiding the cure of atopic dermatitis because silk is able to maintain the moisture balance of the skin, providing a softening and soothing micro-climate next to the skin. Silk is also capable of absorbing and releasing moisture without causing irritation, because the diameter of silk's cylindrical fibers simply increase or decrease as moisture is absorbed or released. Since silk yarns are made up of tiny continuous filaments, delicate skin is not disturbed as the moisture content changes. Silk helps to reduce heavy sweating (common in children affected by atopic dermatitis), as well as minimize the loss of moisture that can aggravate skin dryness and itching. Silk is also naturally elastic. When used in a knitted construction, silk fabrics allow garments to move with the body and to remain closely bound to the skin, thereby reducing friction.

DermaSilk fabrics and apparel also incorporate a topical antimicrobial agent that provides protection to the silk fibers against a broad range of bacteria, germs, molds, and fungus. This antimicrobial treatment inhibits the survival of bacteria on the fabric and is highly effective against Staphylococcus aureus, one of the major factors of worsening atopic dermatitis.

While silk has been proven effective in apparel and body wraps, there are inherent deficiencies in the use of silk fibers, yarns, and fabrics as they relate to therapeutic bedding and patient gowns. Unlike knitted apparel and wraps, hospital articles such as sheets, pillowcases, underpads and patient gowns must withstand continuous use exceeding 8 to 9 hours per day, and extensive laundering and drying. Also, hospital articles are typically subjected to various chemicals and stains associated with personal hygiene, and must withstand much greater stresses and strains associated with sleeping adults. Chloride salts in perspiration and deodorants weaken silk, and alcohol-based products such as hairspray and perfumes, and chemical products like nail polish remover easily damage silk fabrics. Water applied to a stain on silk can set the stain or cause a permanent stain ring. Silk cannot be presoaked in detergents or bleaches, as both will damage silk. Further, silk fabrics cannot be air-dried in the sun, since ultraviolet rays degrade silk. Therefore, silk fabrics are less than ideal for widespread use in therapeutic hospital articles.

An inventor of the present invention developed a new fabric technology for therapeutic bedding which overcame some of DermaSilk's shortcomings by employing synthetic fibers, some of which possess non-round cross-sectional shapes. This fabric has shown great promise in treating mild atopic dermatitis, which is the most common type of eczema. Indeed, on Jan. 25, 2007, the U.S. Food and Drug Administration granted 510(k) clearance on the invention as a Class 1 medical device (registration number K061242) for the treatment of mild atopic dermatitis. Notably, that invention represented the first therapeutic bedding to be granted FDA marketing clearance as a Class 1 medical device for skincare therapy.

While impressive, this FDA marketing clearance was not for use within the context of preventing and treating pressure wounds, because dermatitis and pressure wounds are considered to be different disorders. In fact, one would not expect a dermatitis treatment fabric to provide substantial anti-pressure wound properties, particularly since the fabric itself does not alleviate pressure or otherwise cushion bony prominences.

Since the prevention and treatment of pressure wounds remained a serious medical need with no clear solution, there remained a need for a woven fabric having advantageous antimicrobial, anti-static, wicking and drying properties, which is capable of preventing and treating dermatitis, skin wounds, pressure wounds and other skin ailments. This fabric should be non-abrasive, cost effective, and should be able to withstand many washings, detergents, autoclaving, irradiation, and nightly wear as necessary. There was also a need for hospital articles, including sheets, underpads and patient gowns, constructed of this fabric and suitable for continuous use in a hospital or other care facility.

### SUMMARY OF THE INVENTION

The present invention fulfills one or more of these needs in the art by providing a fabric for bedding, including underpads, and patient gowns, that utilize a unique combination of polymer types and fiber configurations to form woven fabrics that are like silk in terms of aesthetics, smoothness, elongation, and moisture handling, but more durable, more stain resistant, and more suitable for articles such as bedding and patient gowns. Further, the fabric is also superior to cotton-containing fabrics because it dries more quickly and stays smooth when wet.

The fabric includes a woven fabric having warp yarns and filling yarns woven to provide a smooth fabric surface. In the preferred embodiment, one of the warp and filling yarns is at least 40% by weight of the fabric of continuous filament nylon. The other of the warp and filling yarns may be from about 0% to 60% by weight of the fabric of continuous filament polyester or nylon having non-round filament cross sections. Preferably the fabric includes a conductive yarn from about 0% to about 2% by weight of the fabric. Preferably, an antimicrobial substance is topically applied or inherently available in the fabric.

Also, the warp yarns may be 100% nylon, and the filling yarns may be polyester or nylon.

The fabric is preferably woven as a twill weave or plain weave. The warp yarn may be a 40 denier, 34 filament, five twist per inch, continuous filament nylon 6-6 yarn, with the filling yarn a 75 denier, 48 filament, continuous filament textured polyester. In the most preferred embodiment, the warp yarn is a 70 denier, 48 filament, continuous filament textured nylon yarn, the filling yarn a 75 denier, 36 filament, non-round cross-section continuous filament textured polyester, and the conductive yarn is from about 0% to about 2% by weight of the fabric.

Preferably, the continuous filaments have a non-round fiber cross section, such as fibers with a star shaped, clover leaf, trilobal or bowtie cross section. The continuous filaments with non-round fiber cross sections typically have a cross section such that adjacent filaments form wicking channels that enhance moisture wicking and rapid drying.

Also preferably, the fabric is finished to produce a fabric with an elongation greater than about 30% per ASTM D5034-95 test for breaking strength and elongation of textile fabrics.

In the preferred embodiment, the fabric has a soil-release topical finish.

In one aspect, the present invention provides a therapeutic woven fabric for preventing and reducing skin wounds having warp yarns and filling yarns woven to provide a smooth fabric surface and made into a sheet sized to cover a bed. The sheet typically has hems to prevent raveling of the woven fabric. One of the warp and filling yarns is at least 40% by weight of the fabric of continuous filament nylon, the other of the warp and filling yarns is from about 0% to 60% by weight of the fabric of continuous filament polyester or nylon having non-round filament cross sections, and the conductive yarn from about 0% to about 2% by weight of the fabric. Preferably, an antimicrobial substance is topically applied or inherently available in the fabric.

Another aspect of the present invention is to provide a therapeutic woven fabric for use in articles that contact the skin. One of the warp and filling yarns is at least 40% by weight of continuous filament nylon, the other of the warp and filling yarns is from about 0% to 60% by weight of continuous filament polyester or nylon having non-round filament cross sections, and a conductive yarn is from about 0% to about 2% by weight of the fabric. In the preferred embodiment, an antimicrobial substance is topically applied or inherently available in the fabric.

Still another aspect of the present invention is to provide a therapeutic woven fabric with a smooth surface for constructing bedding, including underpads and/or garments such as patient gowns. The warp and filling yarns are continuous and substantially free of hairiness. One of the warp and filling yarns is preferably a continuous filament yarn having a non-round filament cross section such that adjacent filaments form wicking channels, and a conductive yarn is integrated to control static. An antimicrobial substance may be topically applied or inherently available in the fabric.

An invention can also be considered as providing a method of patient therapy including covering the patient's bed with sheets having surfaces that are smooth even when wet; and allowing the patient to rest between the sheets. Preferably, the patient's bed includes an underpad, and the patient is wearing a patient gown, with both the underpad and gown constructed of a fabric having these same properties as the sheets. The surface roughness between the sheets, underpad and patient gown, and the patient's skin is reduced in comparison with the surface roughness that would exist between cotton or polyester/cotton blend fabric sheets and the patient's skin.

An invention can also be considered as providing a method of patient therapy including dressing or providing a patient with a patient gown having smooth surfaces even when wet. Preferably the patient's bed includes bedding, including an underpad, constructed with the same fabric as the patient gown. The surface roughness between the patient gown and the patient's skin is reduced in comparison with the surface roughness that would exist between cotton or polyester/cotton blend fabric sheets and the patient's skin.

Other aspects of the invention provide methods of preventing or treating skin wounds and disorders by dressing or providing a patient with a patient gown constructed of a therapeutic fabric, and replacing the article when it becomes soiled. Alternatively, the article may be replaced according to hospital protocol, for example every 24 hours. Desirably, the therapeutic fabric is constructed from a woven fabric having warp yarns and filling yarns woven to provide a smooth fabric surface, with one of the warp and filling yarns being at least 40% by weight of the fabric of continuous filament nylon, the other of the warp and filling yarns being from about 0% to 60% by weight of the fabric of continuous filament polyester or nylon having non-round filament cross sections, and including a conductive yarn from about 0% to about 2% by weight of the fabric. Preferably the patient is also provided one of at least a bottom bed sheet, top bed sheet, pillowcase, and/or underpad. The article should have an average geometric surface roughness of 1.0 to 1.75 microns when dry as measured by the Kawabata Evaluation System FB4 Surface Tester. Preferably, the article or articles provide an antimicrobial reduction of Escherichia coli, Staphylococcus aureus, and Staphylococcus epidermidis of at least 98.0% after 24-hour contact time.

Another aspect of this invention is a patient gown comprising a textile fabric woven of 100% continuous synthetic yarns, with the fabric finished to achieve antimicrobial reduction of greater than 99% of Staphylococcus aureus and Enterococcus faecalis after 24-hour contact time when tested by the AATCC Test Method 100, having an average surface roughness of about 1.75 microns using the Kawabata Surface Roughness Evaluation, having a moisture wicking rate of greater than 15 mm/minute, and having a drying rate of greater than 1.0% per minute.

In one aspect, the present invention provides a therapeutic woven fabric for constructing an underpad capable of preventing and reducing skin wounds having warp yarns and filling yarns woven to provide a smooth fabric surface, and sized to fit on a bed. The underpad may have hems or seams to prevent unraveling of the woven fabric, include a liquid impermeable bottom layer and/or a fluid absorbing middle layer, and may be at least 34 inches in width along the top edge, at least 42 inches at the bottom edge, and a total length of 65 inches so as to increase protection for patients' knees and feet.

An invention can also be considered as providing a method of patient therapy including covering the patient's bed with an underpad whose top fabric layer has a smooth surface, even when wet; and allowing the patient to rest upon this underpad. Preferably the patient's bed includes at least one other bedding article such as a bottom sheet, top sheet, and/or pillow case, and/or the patient is wearing a patient gown, with the article(s) and patient gown constructed of the same fabric as the underpad's top fabric layer. The surface roughness between the top fabric layer and the patient's skin is reduced in comparison with the surface roughness that would exist between a conventional underpad's top fabric layer constructed of cotton or polyester/cotton blend fabric, and the patient's skin.

Other aspects of the invention provide methods of preventing or treating skin wounds and disorders by providing an underpad to a patient and replacing the underpad when it becomes soiled. Alternatively, the underpad may be replaced according to hospital protocol, for example after 24 hours of use. Desirably, the top fabric layer of the underpad is constructed from a woven fabric having warp yarns and filling yarns woven to provide a smooth fabric surface, with one of the warp and filling yarns being at least 40% by weight of the fabric of continuous filament nylon, the other of the warp and filling yarns being from about 0% to 60% by weight of the fabric of continuous filament polyester or nylon having non-round filament cross sections, and including a conductive yarn from about 0% to about 2% by weight of the fabric. The top fabric layer of the underpad should have an average geometric surface roughness of 1.0 to 1.75 microns when dry as measured by the Kawabata Evaluation System FB4 Surface Tester. Preferably, the top layer of the underpad provides an antimicrobial reduction of Escherichia coli, Staphylococcus aureus, and Staphylococcus epidermidis of at least 98.0% after 24-hour contact time.

Another aspect of this invention is an underpad with a top fabric layer comprising a textile fabric woven of 100% continuous synthetic yards, with the fabric finished to achieve antimicrobial reduction of greater than 99% reduction of Staphylococcus aureus and Enterococcus faecalis after 24-hour contact time when tested by the AATCC Test Method 100, having an average surface roughness of about 1.75 microns using the Kawabata Surface Roughness Evaluation, having a moisture wicking rate of greater than 15 mm/minute, and having a drying rate of greater than 1.0% per minute. Preferably this underpad also includes a liquid impermeable bottom layer and fluid absorbing middle layer, with all three layers secured together.

Other aspects of the invention provide methods of preventing or treating skin wounds and disorders by providing an article to a patient and replacing the article when it becomes soiled. Desirably, the article is constructed from a woven fabric having warp yarns and filling yarns woven to provide a smooth fabric surface, with one of the warp and filling yarns being at least 40% by weight of the fabric of continuous filament nylon, the other of the warp and filling yarns being from about 0% to 60% by weight of the fabric of continuous filament polyester or nylon having non-round filament cross sections, and including a conductive yarn from about 0% to about 2% by weight of the fabric. The articles of this method could be one or more of a bottom bed sheet, top bed sheet, pillowcase, underpad and/or patient gown. The article should have an average geometric surface roughness of 1.0 to 1.75 microns when dry as measured by the Kawabata Evaluation System FB4 Surface Tester. Preferably, the article or articles provide an antimicrobial reduction of Escherichia coli, Staphylococcus aureus, and Staphylococcus epidermidis of at least 98.0% after 24-hour contact time.

These and other aspects of the present invention will become apparent to those skilled in the art after a reading of the following description of the preferred embodiment when considered with the drawings.

### BRIEF DESCRIPTION OF THE DRAWINGS

FIG. 1 is an illustration of a bed having a top sheet and pillow cases constructed of fabric of the present invention;

FIG. 2 is an illustration of a bed having a bottom sheet and underpad constructed of fabric of the present invention;

FIG. 3 depicts a patient wearing an embodiment of the patient gown of the present invention;

FIG. 4 is a schematic representation of an underpad in cross-section;

FIG. 5 is a schematic representation of a patient gown blank;

FIG. 6 is a photomicrograph of yarn whose fibers are star-shaped in cross section;

FIG. 7 is a photomicrograph of yarn whose fibers are cloverleaf-shaped in cross section;

FIG. 8A is a photomicrograph of an embodiment of the fabric of the present invention; and

FIG. 8B is a photomicrograph of conventional hospital bedding fabric.

FIG. 9 shows Table 1, Wicking Comparison;

FIG. 10 shows Table 2, Drying Comparison;

FIG. 11 shows Table 3, Present Reduction (%) at 24-hours contact time; and

FIG. 12 shows Table 4.

### DESCRIPTION OF THE PREFERRED EMBODIMENTS

A therapeutic fabric for the prevention and treatment of skin wounds and disorders is disclosed. As used herein “the fabric” or “the therapeutic fabric” shall refer to this fabric unless the context implies differently. The fabric is used to form articles that contact the skin, such as bedding and patient gowns. Used herein “bedding” shall refer to bottom bed sheets, top bed sheets, pillow cases and/or underpads, and “articles” shall refer to bedding and patient gowns. The therapeutic fabric includes warp yarns and filling yarns woven to provide a smooth fabric surface. In the preferred embodiment, one of the warp or filling yarns is at least 40% by weight of the fabric of continuous filament nylon, and the other of the warp or filling yarns is from about 0% to about 60% by weight of the fabric of continuous filament polyester or nylon having non-round filament cross sections. The most preferred embodiment includes about 1% to about 2% of a conductive yarn to control static electricity.

Preferably, an antimicrobial substance is topically applied or inherently available in the fabric. In the preferred embodiment, an antimicrobial substance such as AEGIS Microbe Shield, manufactured by AEGIS Environments, Inc., is topically applied to the woven fabric in a standard textile finishing operation. The antimicrobial substance is preferably applied at 0.25% to 2.0%-weight of the active ingredient to 100%-weight of the fabric. AEGIS Microbe Shield works well against the following common microbes: Escherichia coli, Staphylococcus aureus, Staphylococcus epidermidis, and Pseudomonas aeruginosa. The antimicrobial substance may also prevent odors in the fabric.

The warp yarns may be 100% nylon, and the filling yarns may be polyester or nylon.

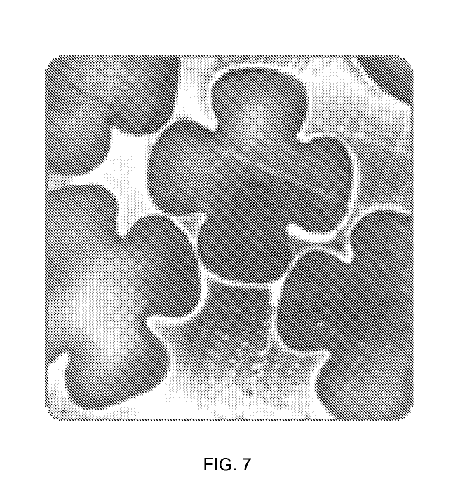

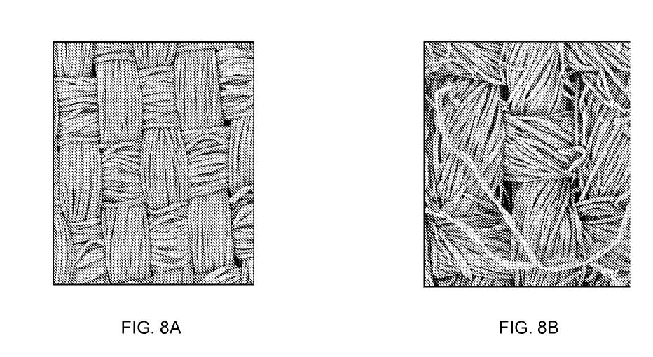

In one embodiment, the fabric is woven as a twill weave (typically a 2X1 twill) or, in another embodiment, as a plain weave. Yarns are woven into fabric constructions that have 80% to 100% coverage. The warp yarn may be a 40 denier, 34 filament, five twist per inch, continuous filament nylon 6-6 yarn, and the filling yarn may be a 75 denier, 48 filament, continuous filament textured polyester. In another embodiment, the warp yarn is a 70 denier, 48 filament, continuous filament, textured nylon, and the filling yarn is a 75 denier, 36 filament, continuous filament, textured polyester. Continuous filament yarns are preferred because those yarns do not have short fibers extending beyond the fabric's planar surface, thereby decreasing irritation to sensitive skin. The smooth fabric surface also accentuates this effect. Continuous filaments forming a smooth surface and short fibers forming an irritating surface are shown in the photomicrographs of FIGS. 8A and 8B, respectively. Warp yarns of about 30 denier to 100 denier, and filling yarns of about 30 denier to 100 denier may be used.

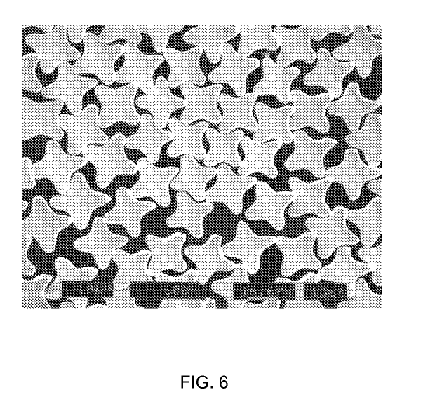

Preferably, the continuous filament has non-round filament cross-sections such as a star-shaped cross section or a clover-leaf cross section. The clover-leaf cross section also improves the fabric's smoothness and softness. Examples of these are seen in FIGS. 6 and 7. With non-round cross sections, adjacent filaments form wicking channels along filament surfaces to promote and enhance moisture transport away from contact with the skin. Thus, moisture more quickly evaporates and dries from the fabric surface, reducing the amount of moisture contacting the skin. As such, the wicking channels also help the user to maintain body temperature by reducing excess sweating.

In the preferred embodiment, nylon is used because it has one of the highest moisture regains of any synthetic fiber. Nylon absorbs moisture, and aids in wicking and evaporation. Although nylon is preferred, polyester can also be used if a durable auxiliary hydrophilic treatment is applied as a post finish.

In the preferred embodiment, the fabric may also contain a soil-release topical finish, such as HydroWick™ NA, from HydroTex USA, Inc., at 40% solids applied at 8% exhausted on the weight of the fabric. Thus, the fabric is able to release stains associated with skin antibiotic creams and ointments, wound drainage, and tissue detritus.

In the preferred embodiment, the fabric may also include a conductive yarn such as carbon, silver or other metal. The conductive yarn is not critical to the therapeutic properties of the present invention, but it is useful in reducing the potential for static electricity which can occur during institutional laundering processes. Specifically, the conductive yarn controls static dissipation by conducting static charges away and preventing a static charge accumulation. The conductive yarn is preferably included as various ends of the warp and/or courses of filling yarns. Preferably, the conductive yarn is about 1% to about 2% by weight of the fabric.

As would be understood by those in the art, when conductive yarn is included in the fabric, the percentage of nylon, non-round filament and conductive yarn would add up to 100%, not 102%. Accordingly, the other yarns in the final fabric will be reduced proportionally. For simplicity herein, however, ranges are set forth as the nylon being at least 40% by weight, the non-round nylon being from about 0% to about 60% by weight, and the conductive yarn being about 0% to about 2% by weight.

The fabric provides exceptional antimicrobial, anti-static, wicking and drying properties, and is capable of preventing and treating dermatitis, skin wounds, pressure wounds and other skin ailments. Moreover, the fabric is non-abrasive, cost effective, and able to withstand many washings, detergents, autoclaving, irradiation, and nightly wear. Accordingly, this fabric is exceptionally well suited for people who bed ridden, receiving rehabilitation, assisted living or nursing care, and particularly those who are exposed to biologicals including sweat, urine, feces, vomitus, wound drainage.

Preferably, the fabric is finished to produce a fabric with an elongation greater than about 30% as measured by ASTM D5034-95. Those of ordinary skill in the art are familiar with suitable finishing technologies to achieve this result.

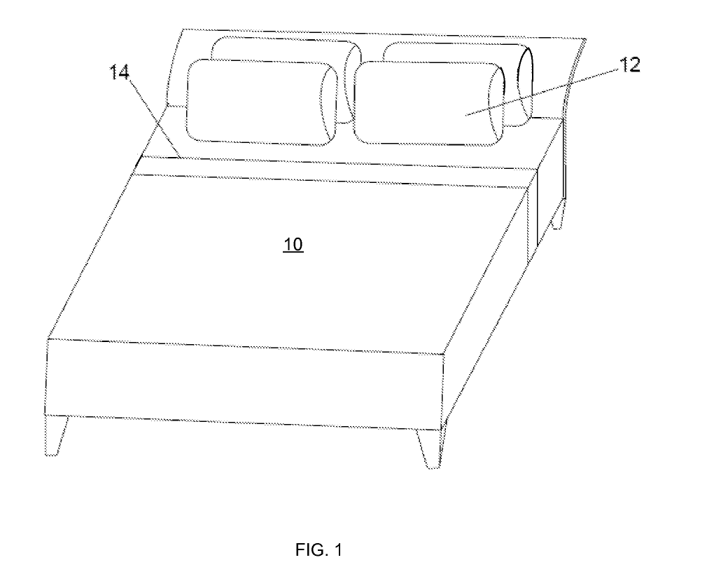

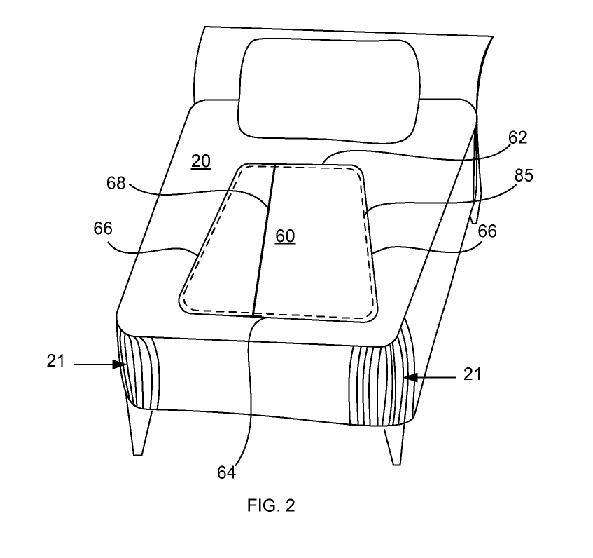

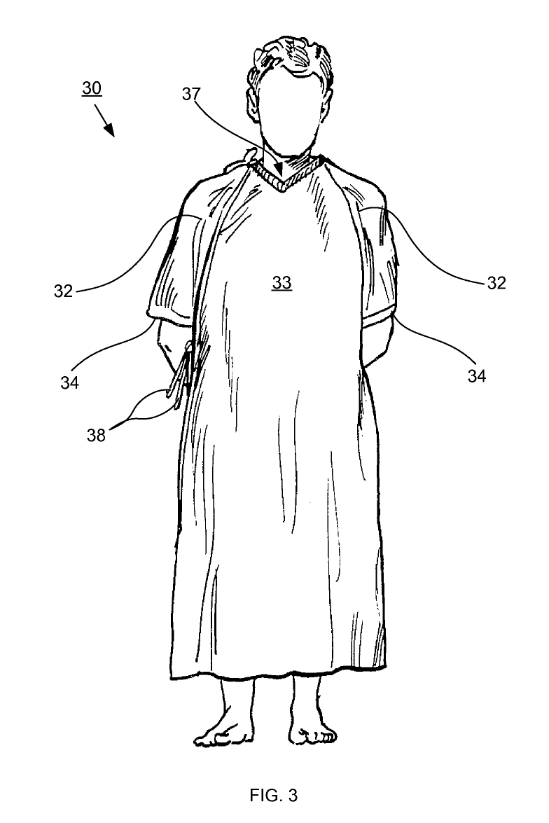

The fabric lends itself to the construction of various articles that come in skin contact including top sheet 10 which includes hem 14 to prevent raveling, and pillowcase 12 also having a hem (not shown), both shown in FIG. 1. As shown in FIG. 2, the fabric may also be used to construct bottom sheet 20, underpad 60, and patient gown 30, as shown in FIG. 3

Referring to FIG. 2, underpad 60 is positioned on bottom sheet 20, which preferably includes fitted corners 21. Typically, a patient (not shown) lays directly on top of underpad 60 with top sheet (not shown) draped over the patient. Underpad 60 is preferably trapezoidally shaped with top edge 62 being less wide than bottom edge 64 when positioned on a bed. Most preferably, underpad 60's top edge 62 is at least 34 inches wide, bottom edge 64 is at least 42 inches wide, and length 68 is approximately 65 inches. These dimensions advantageously provide the pad with sufficient area to extend under the patients' knees and feet, which are particularly susceptible to pressure wounds.

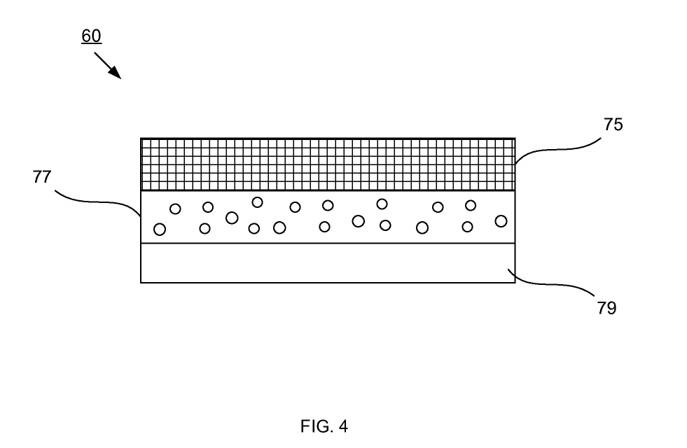

FIG. 4 shows a cross-sectional view of underpad 60 including top fabric layer 75, which is comprised of therapeutic fabrics discussed herein, middle layer 77, and bottom layer 79. Absorbent middle layer 77 is preferably constructed of a nonwoven or foam material. Bottom layer 79 is preferably liquid impermeable, and can be constructed of materials such as vinyl, rubber, polyurethane-coated materials, and other liquid barriers known in the art. Top fabric layer 75, middle layer 77 and bottom layer 79 are preferably secured together to form underpad 60 by perimeter stitching 85, shown in FIG. 2.

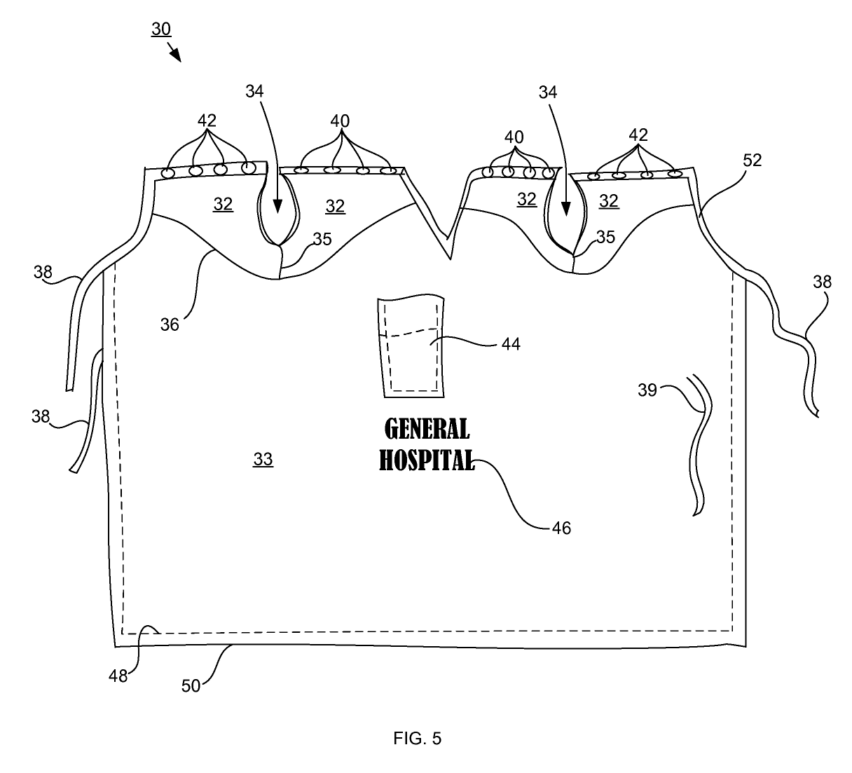

The fabric also lends itself to the construction of patient gown 30, shown in FIG. 3, including arm panels 32 and body 33. FIG. 5 depicts a preferred blank for constructing patient gown 30. Preferably, a continuous piece of therapeutic fabric comprises body 33. There are preferably two sets of two arm panels 32 (for a total of four), all of which are comprised of the therapeutic fabric. Each individual panel 32 is attached to another arm panel 32 via arm hole seams 35. Each pair of two arm panels 32 are attached to body 33 via arm panel seams 36. Arm panels 32 define arm holes 34, through which arms extend when the assembled gown is worn by a patient, as shown in FIG. 3.

Running along the upper edge of arm panels 32 are first fasteners 40 and second fasteners 42, each mating one with another, for example as male and female snaps or buttons, loop and hook attachments, and the like. The innermost arm panels 32 define neck hole 37 (shown in FIG. 3) which may be a v-neck, semi-circular, or the like. Ties 38 secure to one another (such as by tying) behind the patient, as shown in FIG. 3. Alternative means for securing patient gown 30 to the patient include snaps, buttons, loop and hook assemblies, hooks, zippers, adhesives and the like. As shown with respect to the bottom right tie 39 of FIG. 5, tie 39 may be attached to body 33 in a position other than outermost edge 50.

Preferably patient gown 30 includes hem 48 and/or piping 52 to finish edges, pocket 44 for holding small personal or medical items, and identification 46 which indicates name of hospital, department, brand of item or any other desirable indicia.

One comparative example of the fabric is a construction of 84 warp yarns per inch and 96 fill yarns per inch, to produce a fabric which is 2.36 ounces per square yard. The warp yarn is a 70 denier, 49 filament continuous textured nylon, with the primary filling yarn a 75 denier, 36 non-round cross-section continuous filament textured polyester. A secondary filling yarn is a two-ply yarn made by twisting a 25 denier, 3 filament, carbon yarn with a 70 denier, 68 continuous filament polyester yarn, and woven into the fabric at 2.5 picks per inch or 1% of the total weight of the fabric. For purposes of interpreting Tables 1-3 below, this construction shall be referred to as the therapeutic fabric, which is but one embodiment of the present invention.

In contrast, typical hospital bedding fabric is a plain weave in a construction of 120 warp yarns per inch and 74 filling yarns per inch, producing a fabric that is 4.12 ounces per square yard, or 75% heavier than the present invention. The warp yarns are 39/1 spun yarns (equivalent to 136 denier) in a blend of 50% cotton and 50% staple polyester. The filling yarns are 35.6/1 spun yarns (equivalent to 149 denier) in a blend of 50% cotton and 50% staple polyester. These cotton-blend fabrics have no special finishes, moisture handling characteristics, antimicrobial properties, or soil-release properties. For purposes of interpreting Tables 1 and 2, this construction shall be referred to as “Cotton Blend”.

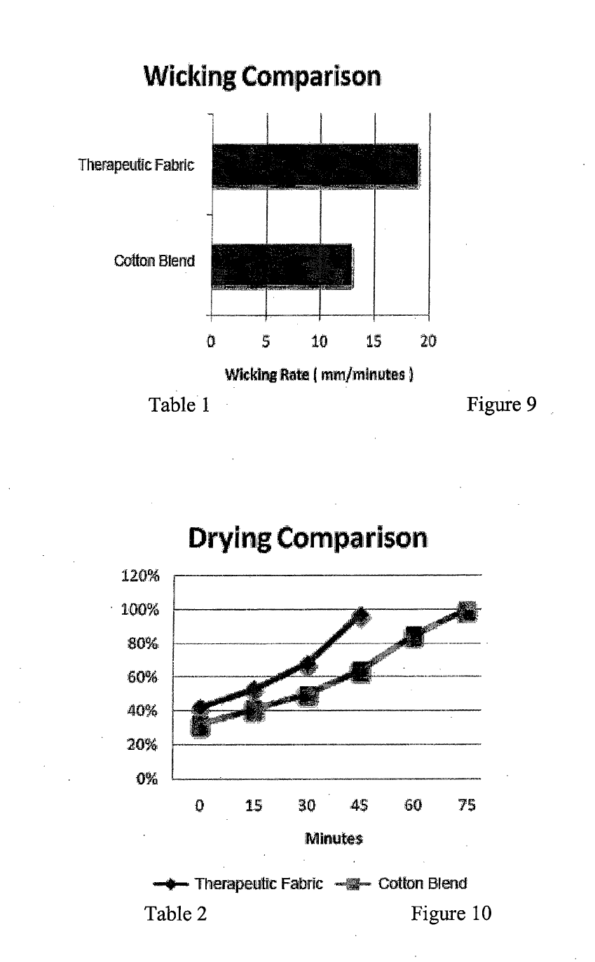

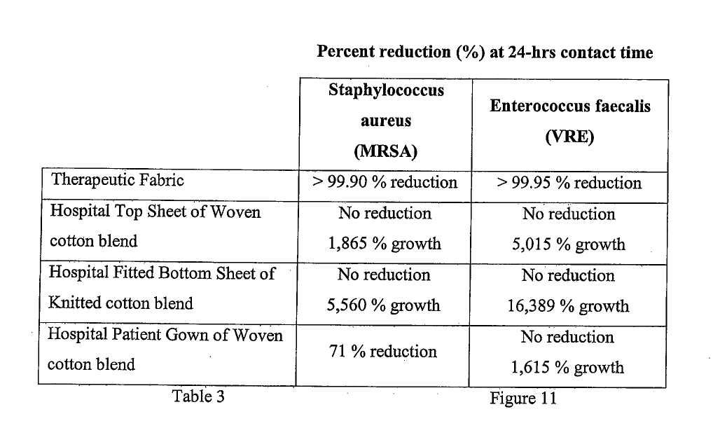

As indicated in the leftmost column of Table 3 (FIG. 11), conventional hospital bedding fabric constructions, not limited to “Cotton Blend” of Tables 1 and 2 (FIGS. 9 and 10), are used for comparison purposes in Table 3 (FIG. 11).

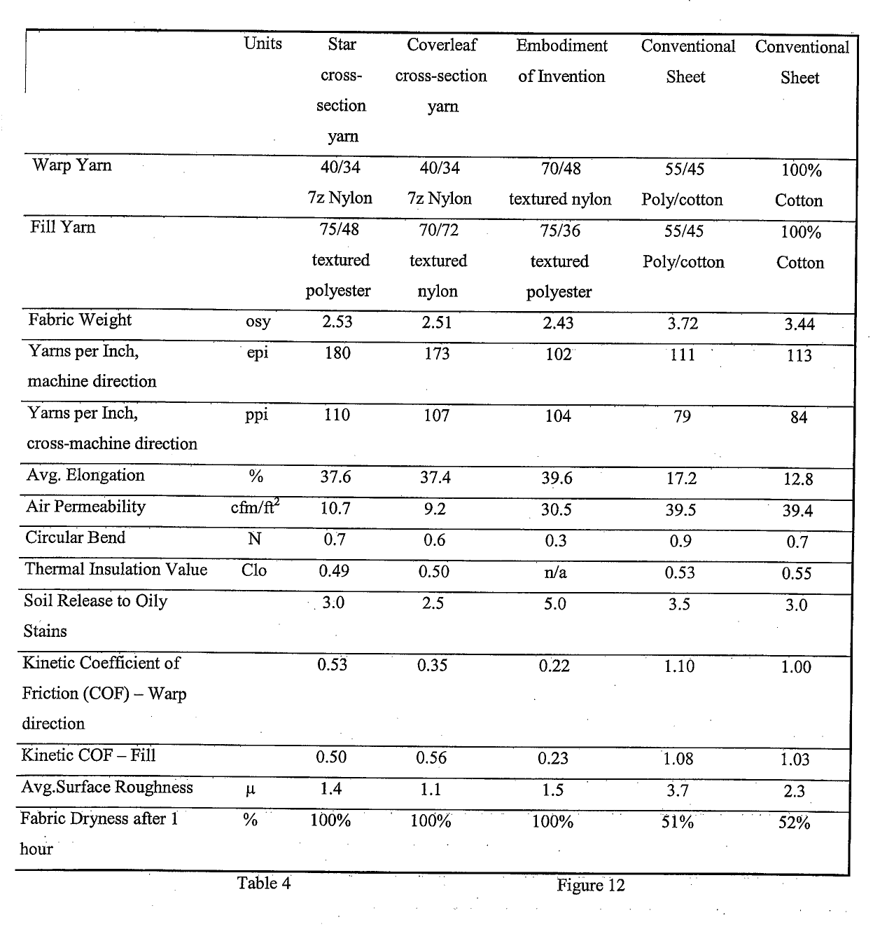

Preferred fabrics have the following properties:
High moisture regain. Nylon, with one of the highest moisture regains of any synthetic fiber, absorbs moisture and aids in wicking and evaporation. This contributes to the superior wicking ability to the present invention, which is demonstrated in Table 1 (FIG. 9).
Excellent moisture transport. Non-round fiber cross sections create channels along fiber surfaces to promote and enhance moisture transport away from contact with the skin. Moisture more quickly evaporates and dries, and thereby reduces the amount of wetness next to the skin. The therapeutic fabric's ability to be greater than 95% dry after 45 minutes is a considerable improvement over the cotton blend, as shown in Table 2 (FIG. 10).
Minimal friction with the skin. Continuous-filament yarns have no short fibers extending beyond the fabric's planar surface to irritate sensitive skin. A smooth fabric surface accentuates this effect. Preferably, the fabric has an average geometric roughness of less than about 1.7 microns as measured by the Kawabata Evaluation System FB4 Surface Tester. Photomicrographs of the present invention and cotton blend, FIGS. 8A and 8B respectively, demonstrate their substantial surface differences.
Substantial antimicrobial properties. Per AATCC Test Method 100, the preferred embodiment of the present invention demonstrates substantial microcidal efficacy (>99.9%) against Staphylococcus aureus, and Enterococcus faecalis in a variety of articles, while various conventional hospital fabrics do not. This is shown in Table 3 (FIG. 11).
A good degree of stretch and recovery. Such fabrics help bed sheets to fit tighter and thereby reduce wrinkles that cause skin irritation. Such fabrics also better conform to the body and reduce the shear forces on sensitive skin. Preferably, the fabric is finished to produce a fabric with an elongation greater than about 30% as measured by ASTM D5034-95. The superior elongation properties are demonstrated in Table 4 (FIG. 12).
Durability to extended laundering and drying. Such fabrics will not lose fibers during laundering (in comparison with cotton blends), and are not afflicted with fiber pills that further irritate skin.
Ability to withstand high wash temperatures and the use of harsh detergents.
Ability to release stains associated with skin antibiotic creams and ointments, wound drainage, and tissue detritus. This is demonstrated in Table 4 (FIG. 12).

The data relating to various properties of star and cloverleaf cross-sectional yarns, and one embodiment of the invention are compared with typical 55/45 polyester/cotton and 100% cotton bedding fabrics in Table 4 (FIG. 12).

While the physical properties of the fabric are significantly superior to conventional fabrics, the inventors, textile experts and medical experts were surprised by the dramatic clinical results obtained when the therapeutic fabric was tested in a clinical study in a hospital on actual patients. In short, patients who used articles constructed of the present invention fabric were significantly less likely to develop pressure wounds, and significantly less likely to be discharged with pressure wounds, versus patients who used standard hospital-issue articles.

In the study, 307 patients in the Medical Renal Ward of a regional hospital were enrolled in the study. This patient population was selected because of their affliction with multiple co-morbidities in addition to renal failure, and because they were at high risk for pressure wound development. The purpose of the study was to evaluate 1) the reduction in friction, shear, and pressure damage to the skin, and 2) the reduction in maceration and skin fold breakdown, where the bedding, including underpads, and patient gowns help protect moist skin folds caused by sweat and incontinence. Hospital bedding, including underpads, and patient gowns made with the fabric invention (hereafter referred to as the “invention ensemble”) were compared with the incumbent cotton-blend sheets, including underpads, and patient gowns used in the hospital (hereafter referred to as the “standard ensemble”).

For the duration of the trial, all newly admitted patients received either the invention ensemble or standard ensemble, depending on when they were admitted. Specifically, all patients admitted during the first eight weeks of the study used the standard ensemble, patients admitted during the second eight weeks received the invention ensemble, and patients admitted during the final seven weeks received the standard ensemble. The 307 test subjects were statistically similar with respect to demographic factors such as weight, age, and co-morbidities. Their average length of stay was just over five days. Study data was analyzed used the t-test statistical method to determine whether two sample means were equal. Statistical significance was indicated when p<0.05; that is, when there was a 95% chance or greater that the differences in the means were not due to chance. The key findings are given below.
61.7% fewer wounds were developed using the invention ensemble, as compared with the standard ensemble. The results had a p-value of 0.0137, and were considered statistically significant.
39.6% fewer wounds were found at patient discharge using the invention ensemble, as compared with the standard ensemble. The results had a p-value of 0.0315, and were considered statistically significant.
Normalizing the data to account for patients' length of stay in the renal ward, 56.8% fewer wounds per 1,000 patient days were developed using the invention ensemble, as compared with the standard ensemble. The results had a p-value of 0.0137, and were considered statistically significant.
32.0% fewer wounds per 1,000 patient days were found at discharge using the invention ensemble, as compared with the standard ensemble. The results had a p-value of 0.0315, and were considered statistically significant.

The foregoing results demonstrated that the inventors have successfully conceived and reduced to practice an effective therapy for the prevention and treatment of pressure wounds in healthcare settings.

To prevent or treat skin wounds or disorders, a patient is provided one or more articles which are constructed of the fabric. Suitable articles include a bottom bed sheet, top bed sheet, pillow case, underpad and/or patient gown. These articles are considered to be in “substantial skin contact” with the patient insofar as it is foreseeable that the skin of a bed-ridden patient will come in contact with the article within a 24 hour period in the hospital. The article or articles should be replaced when they become undesirably moist or soiled, or otherwise according to standard hospital procedures which require, for example, complete bedding changes every 24 hours.

Pressure wounds may also be prevented or cured by obtaining a bottom sheet, top sheet, pillow case, underpad, and/or patient gown marketed as a pressure wound preventative or curative, providing that article for use, and optionally providing instructions or information. “Marketed” as used herein, refers to the written or electronic dissemination of information about the sheet, pillowcase, underpad or gown.

The invention also covers a method of preventing and/or treating pressure wounds by obtaining an article marketed as a pressure wound preventative, employing the article in connection with the care of a patient would subject to the development of pressure wounds under conventional therapies, and monitoring the patient for the development of pressure wounds. This article may be replaced when undesirably moist or soiled, may have an average geometric surface roughness of about 1.0 to 1.75 microns when dry, and/or may have a conductive yarn.

The invention also covers a method of preventing and treating pressure wounds by marketing at least one article as a pressure wound preventative and treatment, providing the article to the healthcare provider, and providing information to the healthcare provider on use of the article to prevent or treat pressure wounds.

The invention also covers weaving a fabric, applying an antimicrobial to the fabric, cutting and sewing the fabric to form an article, promoting the sale of the article as a pressure wound preventative or treatment, and providing the article to a health care provider. This article may include a conductive yarn.

Certain modifications and improvements will occur to those skilled in the art upon a reading of the foregoing description. For example, the invention may be carried out with weaves other than plain weaves or twill weaves. The weaves of the invention create a smooth flat surface, without any three-dimensional surface structures that might unduly abrade skin. Other weaves that could be substituted include satin, sateen, or duck weaves. It should be understood that all such modifications and improvements have been deleted herein for the sake of conciseness and readability but are properly within the scope of the following claims.

1. A patient gown comprising textile fabric woven of 100% continuous synthetic filament yarns, said fabric:
finished to achieve antimicrobial efficacy of greater than 99% reduction of Staphylococcus aureus for 24-hours per AATCC Test Method 100;
finished to achieve antimicrobial efficacy of greater than 99% reduction of Enterococcus faecalis for 24-hours per AATCC Test Method 100;
having an average surface roughness of about 1.75 microns using the Kawabata Surface Roughness component;
having a moisture wicking rate of greater than 15 mm/minute; and
having a drying rate of greater than 1.0% per minute.

2. The patient gown according to claim 1 wherein said fabric includes a conductive yarn at about 1% to about 2% by weight of fabric, said conductive yarn capable of controlling static dissipation.

3. The patient gown according to claim 1 wherein the filling yarns of said fabric are 100% polyester.

4. The patient gown according to claim 1 wherein said fabric is 100% nylon.

5. The patient gown according to claim 1 wherein said fabric includes a durable stain-release finish.

6. A patient gown capable of preventing and reducing skin wounds comprising a garment to be worn by a patient made of a fabric comprising:
warp yarns, filling yarns and conductive yarn woven to provide a smooth fabric surface with one of the warp or filling yarns being at least about 40% by weight of the fabric of continuous filament nylon, and the other of the warp or filling yarns being from about 0% to about 60% by weight the fabric of continuous filament polyester or nylon having non-round filament cross sections, and the conductive yarn from about 0% to about 2% by weight of the fabric; and
an antimicrobial substance topically applied or inherently available in the fabric.

7. The gown according to claim 6, wherein the warp yarns are 100% nylon.

8. The gown according to claim 6, wherein the filling yarns are 100% polyester.

9. The gown according to claim 6, wherein the filling yarns are 100% nylon.

10. The gown according to claim 6, wherein the fabric is woven as a plain weave.

11. The gown according to claim 6, wherein the warp yarn is a 70 denier, 48 filament, textured, continuous filament nylon yarn.

12. The gown according to claim 6, wherein the filling yarn is a 75 denier, 36 filament, continuous filament textured polyester.

13. The gown according to claim 6, wherein the continuous filament having non-round fiber cross sections has a star shaped cross section.

14. The gown according to claim 6, wherein the continuous filament having non-round fiber cross sections has a clover leaf cross section.

15. The gown according to claim 6, wherein the continuous filament having non-round fiber cross sections has a cross section such that adjacent filaments form wicking channels.

16. The gown according to claim 6, wherein the conductive yarn is carbon.

17. The gown according to claim 6, wherein the conductive yarn is 25/3 carbon yarn plied with 70/68 textured polyester yarn.

18. The gown according to claim 6, further comprising a soil-release topical finish.

19. A method of preventing or treating skin wounds and disorders comprising the acts of:
providing a patient gown constructed of therapeutic fabric having a smooth finish to a patient, whereby the patient gown is in substantial skin contact with the patient, and;
replacing the patient gown when it becomes undesirably moist or soiled.

20. The method of claim 19 wherein the act of providing a patient gown of therapeutic fabric includes the act of providing woven synthetic therapeutic fabric having warp yarns and filling yarns, with one of the warp or filling yarns being at least 40% by weight of the fabric of continuous filament nylon, the other of the warp or filling yarns being from about 0% to about 60% by weight of the fabric of continuous filament polyester or nylon having non-round filament cross sections, a conductive yarn, and an antimicrobial substance topically applied or inherently available in the fabric.

21. The method of claim 20 wherein the act of providing a patient gown of therapeutic fabric with yarns with non-round filament cross sections includes providing fabric of yarns with filaments with a star shaped cross section.

22. The method of claim 20 wherein the act of providing a patient gown of therapeutic fabric with yarns with non-round filament cross sections includes providing fabric of yarns with filaments with a cloverleaf shaped cross section.

23. The method of claim 20 further comprising wicking moisture along channels between filaments of the yarn having non-round filament cross sections.

24. The method of claim 19 wherein the act of providing a patient gown of therapeutic fabric includes the act of providing a gown made of fabric, the smoothness of which is quantified by the fabric having an average geometric surface roughness of about 1.0 to 1.75 microns when dry as measured by the Kawabata Evaluation System FB4 Surface Tester, between the therapeutic fabric and the patient's skin.

25. A method of making a patient gown comprising the acts of:
obtaining a fabric having warp yarns and filling yarns which provide a smooth fabric surface, with one of the warp or filling yarns being at least 40% by weight of the fabric of continuous filament nylon, the other of the warp or filling yarns being from about 0% to about 60% by weight of the fabric of continuous filament polyester or nylon having non-round filament cross sections;
cutting the fabric according to a pattern to form a blank; and
sewing the blank to form a patient gown.

26. The method according to claim 25 wherein the act of obtaining a fabric having warp yarns and filling yarns includes the act of obtaining a woven fabric having warp yarns and filling yarns.

27. The method according to claim 25 wherein the act of obtaining a fabric having warp yarns and filling yarns includes performing at least one act selected from the group consisting of producing, procuring and purchasing.

28. A patient gown as claimed in claim 1 wherein the fabric is woven with a plain weave.

29. A method as claimed in claim 19 wherein providing includes providing the gown of a fabric that is woven with a plain weave.

30. A method as claimed in claim 25 wherein obtaining a fabric includes obtaining a fabric having warp yarns and filling yarns that are woven with a plain weave
